(19) United States
(12) Patent Application Publication
(10) Pub. No.: US 2009/0312769 A1
(43) Pub. Date: Dec. 17, 2009

(54) STYLET FOR STIMULATING MEDICAL IMPLANTS

(75) Inventors: Fysh Dadd, Lane Cove (AU); Claudiu Treaba, Richmond (GB)

Correspondence Address: CONNOLLY BOVE LODGE & HUTZ LLP, 1875 EYE STREET, N.W., SUITE 1100, WASHINGTON, DC, 20006, US

(73) Assignee: COCHLEAR LIMITED, Lane Cove (AU)

(21) Appl. No.: 12/332,338

(22) Filed: Dec. 10, 2008

(30) Foreign Application Priority Data
Dec. 10, 2007 (AU) 2007906688

(51) Int. Cl. A61F 11/04; A61N 1/05

(52) U.S. Cl. 606/129; 607/137

(57) ABSTRACT

An elongate stylet for removable insertion into a lumen of a stimulating medical implant, comprising a plurality of contiguous longitudinal regions, wherein a cross-sectional area of at least some of the regions are different.

### CROSS-REFERENCE TO RELATED APPLICATIONS

This application claims priority from Australian Provisional Patent Application 2007906688 entitled “Stylet For a Medical Implant”, filed on 10 Dec. 2007.

### BACKGROUND

1. Field of the Invention

The present invention relates generally to stimulating medical implants, and more particularly, to a stylet for use with stimulating medical implants.

2. Related Art

The use of medical devices to provide therapy to individuals for various medical conditions has become more widespread as the therapeutic benefits such devices provide become more widely appreciated and accepted throughout the population. In particular, implantable medical devices that provide stimulation, such as pace makers, auditory brain stem implants (ABI), Functional Electrical Stimulation (FES) devices, spinal cord stimulators, cochlear implants, and other medical devices, have been successful in performing life saving, lifestyle enhancement or other therapeutic functions for many individuals.

One such type of stimulating medical implant is a Cochlear™ implant system (commonly referred to as Cochlear™ devices, Cochlear™ implants and the like; “cochlear implant” herein). Cochlear implants provide hearing sensations to individuals suffering from severe to profound hearing loss. Hearing loss in such individuals is typically due to the absence or destruction of the hair cells in the cochlea which transduce acoustic signals into nerve impulses. Cochlear implants essentially simulate the cochlea hair cells by directly delivering electrical stimulation to the auditory nerve fibers. This causes the brain to perceive a hearing sensation resembling the natural hearing sensation normally delivered to the auditory nerve.

### SUMMARY

In one aspect of the present invention, an elongate stylet for removable insertion into a lumen of a stimulating medical implant, comprising: a plurality of contiguous longitudinal regions, wherein a cross-sectional area of at least some of the regions are different.

In another aspect of the present invention, a stylet comprising a handle, a tip, and a main body therebetween, for use in a medical implant, wherein a cross sectional area of the main body of the stylet is reduced at a region of the stylet that requires less bending strength than other regions of the stylet.

In a further aspect of the present invention, a kit comprising: a stimulating medical implant having a lumen; and an elongate stylet for removable insertion into a lumen of the stimulating medical implant, the stylet having a plurality of contiguous longitudinal regions, wherein a cross-sectional area of at least some of the regions are different.

### DETAILED DESCRIPTION

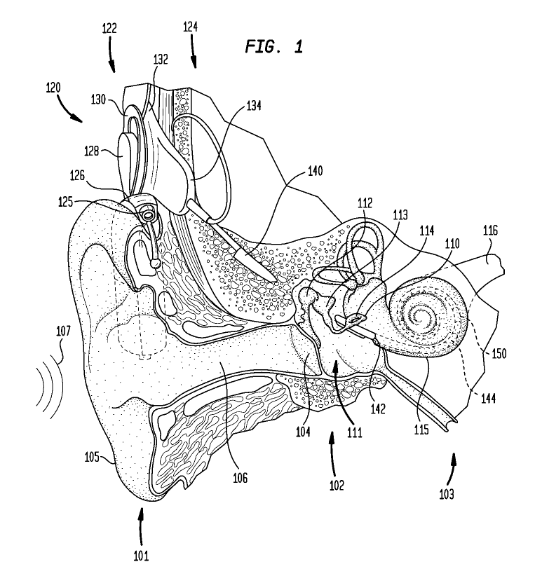

FIG. 1 is a perspective view of an exemplary prosthetic hearing device, a cochlear implant, in which embodiments of the present invention may be advantageously implemented. In fully functional human hearing anatomy, outer ear 101 comprises an auricle 105 and an ear canal 106. A sound wave or acoustic pressure 107 is collected by auricle 105 and channeled into and through ear canal 106.

Disposed across the distal end of ear canal 106 is a tympanic membrane 104 which vibrates in response to acoustic wave 107. This vibration is coupled to oval window or fenestra ovalis 110 through three bones of middle ear 102, collectively referred to as the ossicles 111 and comprising the malleus 112, the incus 113 and the stapes 114. Bones 112, 113 and 114 of middle ear 102 serve to filter and amplify acoustic wave 107, causing oval window 110 to articulate, or vibrate. Such vibration sets up waves of fluid motion within cochlea 115. Such fluid motion, in turn, activates tiny hair cells (not shown) that line the inside of cochlea 115.

Activation of the hair cells causes appropriate nerve impulses to be transferred through the spiral ganglion cells and auditory nerve 116 to the brain (not shown), where they are perceived as sound. In deaf persons, there is an absence or destruction of the hair cells. A cochlear implant such as cochlear implant 120 is utilized to directly stimulate the ganglion cells to provide a hearing sensation to the recipient.

FIG. 1 also shows how a cochlear implant 120 is positioned in relation to outer ear 101, middle ear 102 and inner ear 103. Cochlear implant 120 comprises external component assembly 122 which is directly or indirectly attached to the body of the recipient, and an internal component assembly 124 which is temporarily or permanently implanted in the recipient. External assembly 122 comprises microphone 125 for detecting sound which is outputted to a behind-the-ear (BTE) speech processing unit 126 that generates coded signals. The codes signals are provided to an external transmitter unit 128, along with power from a power source such as a battery. External transmitter unit 128 comprises an external coil 130 and, preferably, a magnet (not shown) secured directly or indirectly in external coil 130.

Internal components 124 comprise an internal receiver unit 132 having an internal coil (not shown) that transcutaneously receives the power and coded signals from external assembly 122, and provides such signals to a stimulator unit 134. In response to the coded signals, stimulator 134 applies stimulation signals to cochlea 115 via an implanted electrode assembly 140. Electrode assembly 140 enters cochlea 115 at cochleostomy region 142 or through oval window 110, and has an array 144 of one or more electrodes 150 positioned to be substantially aligned with portions of tonotopically-mapped cochlea 115. The delivery of stimulation signals at various locations along cochlea 115 causes a hearing percept representative of the received sound 107.

The present invention has application to any stimulating medical implant which has a lumen configured to receive a stylet for controlling the positioning, configuration, or other aspect of the implant. As an example, one type of medical device which may advantageously utilize the present invention is a stylet utilized to implant an electrode assembly of a cochlear implant.

The stylet and lumen have contact surfaces that slide relative to each other. Such relative movement may occur during operation, implantation, explantation or otherwise, and may be mechanically or manually induced and controlled. Also, one or both of the contacting components may be temporarily or permanently implanted, or utilized in the implantation, operation or explantation of any other implanted medical device component.

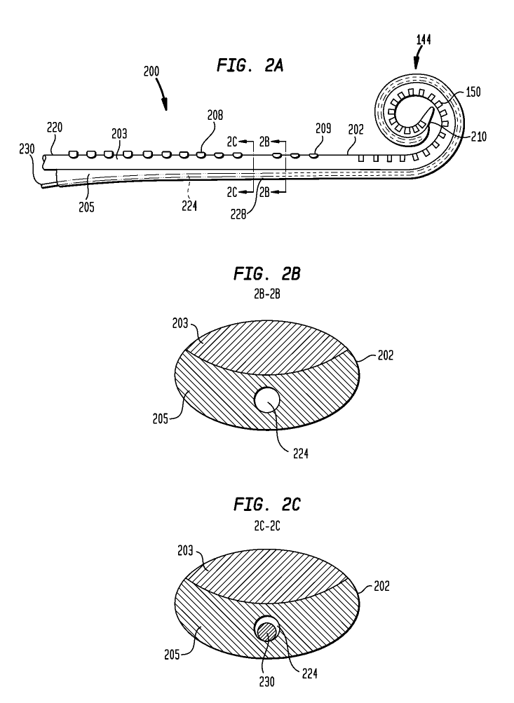

FIG. 2A is a side view of an embodiment of electrode assembly 140 in accordance with one embodiment of the present invention, referred to herein as electrode assembly 200. FIG. 2B is a cross-sectional view of electrode assembly 200 taken along section line 2B-2B in FIG. 2A, while FIGS. 2C is a cross-sectional view of the electrode assembly taken along section line 2C-2C in FIG. 2A.

Electrode assembly 200 comprises a carrier member 202 on which array 144 of electrodes 150 is disposed. As noted, each electrode 150 is constructed and arranged to deliver a stimulating signal to a particular region of cochlea 115. The stimulation signal may be electrical, optical or other form of energy.

It has been found that for electrical stimulation the magnitude of the currents flowing from electrodes 150, and the intensity of the corresponding electric fields, are a function of the distance between electrodes 150 and the modiolus (not shown) of cochlea 115. If this distance is relatively great, the threshold current magnitude must be larger than if this distance is relatively small. Moreover, the current from each electrode 150 may flow in a number of directions, and the electrical fields corresponding to adjacent electrodes may overlap, thereby causing cross-electrode interference. To reduce such adverse effects, it is advisable to maintain a minimal distance between carrier member 202 and the modiolus. This is best accomplished by providing carrier member 202 in a shape which generally follows the shape of the modiolus, or inside wall of cochlea 115. This increases the effectiveness of the delivery of electrical stimulation to auditory nerve 116.

In this exemplary embodiment, to position electrodes 150 adjacent the inside wall of cochlea 115, carrier member 202 adopts a curled or spiral position immediately following implantation into cochlea 115. It is also desirable that carrier member 202 be shaped such that the insertion process causes minimal trauma to the sensitive structures of cochlea 115. As such, carrier member 202 is manufactured to be pre-curved. Specifically, carrier member 202 is manufactured to have a spiral configuration; that is, one or more concentric circles that approximate the curvature of cochlea 115.

Although not shown, different regions of carrier member 202 have inherent bending forces that are different. As such, certain longitudinal regions naturally curve more than others, and some regions may not curve at all.

Usually carrier member 202 is held in a generally straight configuration at least during the initial stages of the insertion procedure, conforming to the natural shape of cochlea 115 once implantation is complete. To have carrier member 202 assume a generally straight configuration, a lumen 224 is provided in the carrier member, as shown in FIGS. 2A-2C. In the illustrative application of carrier member 202, lumen 224 extends through a substantial portion of the length of carrier member 202. It should be appreciated that this is just one embodiment and that the lumen may take on many forms. For example, in another embodiment, carrier member 202 includes a non-communicative lumen that extends through a portion of carrier member 202, as described in U.S. patent application Ser. No. 11/268,592, which is hereby incorporated by reference herein. In a further embodiment, the lumen is defined by a series of concentric circles formed along a surface of carrier member 202.

Lumen 224 is configured to receive a stiffening element 230 commonly referred to in the context of prosthesis hearing implants as a stylet. Although such reference is used in connection with prosthetic hearing devices, it should be appreciated that the term “stylet” is not limiting to any particular application or configuration.

Prior to implanting carrier member 202, stylet 230 is inserted into lumen 224 to maintain electrode array 200 in a substantially straight configuration. While electrode assembly 200 is inserted through cochleostomy 142 or oval window 110, a surgeon biases forward carrier member 202 on stylet 230 to allow carrier member 202 to return to its spiral configuration and, in doing so, to follow the curvature of cochlea 115. In other words, during insertion, stylet 230 is withdrawn from lumen 224 thereby allowing carrier member 202 to return to its pre-curved configuration. A shown by the cross-sectional views of FIG. 2A and FIG. 2B, stylet 230 is partially inserted in lumen 224.

In one embodiment, the technique for implanting electrode assembly 200 is the Advance Off-Stylet™ technique for the Contour™ Advance electrode (previously referred to as the Contour™ Electrode with Softip). In a another embodiment, electrode assembly 200 includes a Contour™ Advance Electrode, also described as Contour™ Electrode with Softip, Modified Tip, or Ski Tip. In another embodiment, the stylet is an Arrow Stylet, Surgical Stylet, or Surgical Ball Stylet. In these and other stylets and electrode carrier members, the stylet is removably inserted into the lumen of the carrier member prior to implantation, and is removed from the carrier member during implantation.

Because implantation of an electrode assembly is a delicate procedure that requires the surgeon to use precise touch and control, any interference may increase the risk of injury to the recipient. At times the force required to withdraw a stylet from a carrier member is greater than anticipated, which may adversely affect a surgeon's tactile control during implantation. For example, as a stylet is removed from a carrier member lumen, damage to delicate structures in the cochlea is possible if an increased withdrawal force causes the surgeon to implant the carrier member at an inappropriate rate or orientation. Likewise, injury to the recipient may occur if the surgeon does not withdraw the stylet at the appropriate end point or at the proper rate during insertion of the carrier member.

It has been observed that static friction is unproportionally larger than the kinetic or dynamic friction (“dynamic friction” herein) between the stylet and carrier member. Thus, although a larger force is required to induce motion, relatively less force is required to maintain such motion. Of the various factors that may contribute to static friction, atomic bonding of the stylet and carrier member is a significant contributor. In many instances the stylet is inserted into the carrier member when the components are manufactured and/or stored for future use. This continued stationary contact of the stylet and carrier member facilitates the atomic bonding of the stylet and carrier member. This bonding is also significant because the pre-curved carrier member presses the lumen against the stylet. This increased compression force also facilitates atomic bonding. Furthermore, the surface area of the contact surfaces may be significant due to the length of the carrier member and stylet. The greater surface area increases static friction due to such atomic bonding. There have been a number of attempts to solve this problem of a high stylet withdrawal force. These include parylene coated stylets as described in US Patent Application No. 20070127745, and Teflon lumens as described in US Patent Application No. 2004/0220651.

A pre-curved electrode assembly is designed to have enough stored elastic energy which exerts a contractive or bending force tending to restore the lead to its original curved shape to facilitate adequate shape retention. During insertion, the electrode assembly must be held in a straight enough configuration to facilitate easy and thus minimally traumatic insertion. The stylet should be strong enough to hold the electrode straight against the stored elastic energy that manifests itself as the noted contractive or bending force.

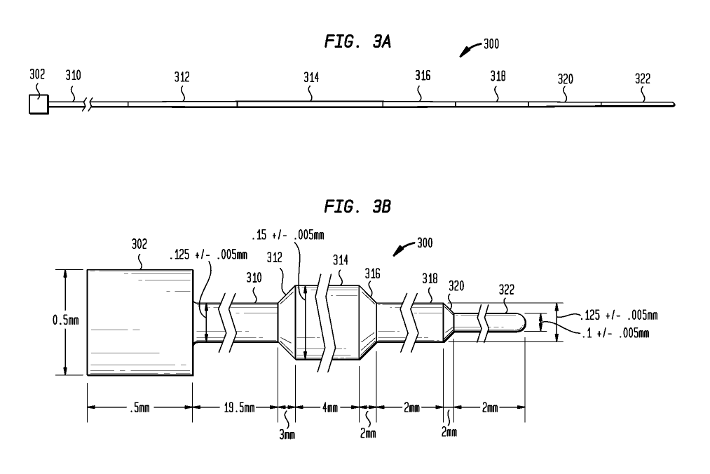

FIG. 3A is a side view of an embodiment of the stylet illustrated in FIGS. 2A-2C, referred to herein as stylet 300. FIG. 3B is a side view of stylet 300 depicted with disproportional dimensions to facilitate understanding of the invention. As noted, stylet 300 comprises a plurality of contiguous longitudinal regions 310, 312, 314, 316, 318, 320 and 322, wherein a cross-sectional area of at least some of the regions are different.

In this exemplary application of a cochlear implant electrode assembly, stylet 300 has a cross-sectional shape of a circle, as shown in FIG. 2C. Alternatively, stylet 300 may have an oval or rectangular cross-sectional shape. Other cross-sectional shapes may be implemented as well. Generally, and in these exemplary embodiments, the cross-sectional shape of the different regions of the stylet is substantially the same. Similarly, the material and, with the exception of the tip region 322, the manufacturing process forming the regions of electrode assembly 300 is substantially the same. As such, in certain embodiments, an increase in the cross-sectional area of the stylet results in an increase in the volume of material and a greater surface area of that region of the stylet. As such, a longitudinal region having a greater cross-sectional area has a greater stiffness and, therefore, a greater resistance to bending forces. Also, the greater surface area provides more potential contact surface area between stylet 300 and the electrode assembly lumen. This, in turn, increases the static and dynamic friction for that region.

Thus, the cross sectional area, or more generally, the volume and contacting surface area, of different longitudinal regions of a stylet may be set so that the bending strength of the stylet is maximized where it is needed. Other regions many be reduced where possible such that a desired withdrawal force, which is closely linked to the fit between the lumen and stylet, is reduced (via reduction in cross section).

In FIG. 3B, there is shown stylet 300, with tip 322, handle 302, and a main body having varying cross-section and cross sectional areas, provided by regions of varying diameter along its length. It will be appreciated that FIG. 3B is not drawn to scale, and that the various features are grossly exaggerated for clarity of explanation. In more accurate proportion, these features will be essentially imperceptible. In contrast, FIG. 3A is a more realistically-representative view of stylet 300.

In FIG. 3B, there is shown rounded tip. It can be seen that a first region 322 of stylet 300 between about 0 mm and about 2 mm from the tip has a diameter of about 0.1 mm. This is in one form, a short, thin, annealed length, and made soft for initial insertion forces.

A second region 318 of stylet 300 between about 4 mm and about 6 mm from the tip has a diameter of about 0.125 mm. This is a slightly thicker region to hold the electrode substantially straight for insertion.

A third region 314 of stylet 300 between about 8 mm and about 12 mm from the tip has a diameter of about 0.15 mm. This is a relatively thick region, and has been maximised to assist in holding the electrode assembly as straight as possible in the region with the highest elastic bending force. This region therefore has a larger cross sectional area than the rest of stylet 300, beyond handle 302.

A fourth region 310 of stylet 300 between about 15 mm and about 34.5 mm from the tip has a diameter of about 0.125 mm. This is a thinner region, as there is little or no precurve elastic force of the precurved lead to compete against. Stylet 300 is therefore reduced in diameter to reduce the stylet withdrawal force.

In another aspect, it can be seen that the modified stylet 300 will provide a benefit if the regions that are required to withstand less bending force are caused to have a smaller cross sectional area than that or those regions required to withstand greater or greatest bending force. For example, with reference to FIG. 4, it may be determined that the region that must withstand the greatest bending force is that between about 8 mm and 12 mm form the tip. Accordingly, all other regions may be formed with reduced cross sectional area.

The location and degree of bending force required to be withstood for a particular application will be apparent to the person of ordinary skills in the art, or at least easily determined using conventional and known means.

As can be seen in FIG. 3B, in one form, there is provided a first transition region 320 between first region 322 and second region; 318, a second transition region 316 between second region 318 and third region 314, and a third transition region 312 between third region 314 and fourth region 310. It should be appreciated that such transition regions may have many configurations. Preferably, the transitions are gradual and there are minimal abrupt edges.

In one form, at least one of the first transition region, the second transition region and/or the third transition region is tapered. This assists in reducing stress concentrators caused by sharp transitions from one region to another. However, it will be understood that one, more or all of these transition regions could be removed, to provide for immediate change from one region to another.

FIG. 4 is a side view of an alternative stylet 400, in which the main body of the stylet is substantially uniform, except for the larger region, having greater cross sectional area, to cater for increased bending force in that region.

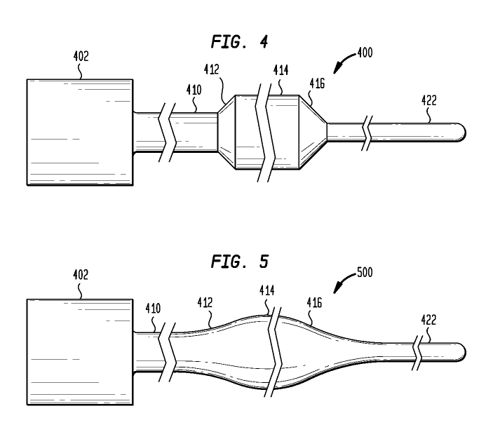

FIG. 5 shows yet a further alternative, in which the main body increases to the maximum diameter in a smooth and continuous fashion, rather than in discrete regions.

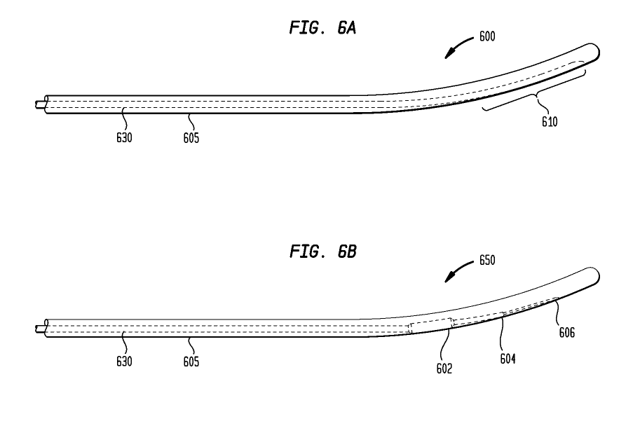

FIG. 6A illustrates a lumen 605 of an electrode assembly while in its substantially straight configuration prior to implantation. A conventional stylet 620 is inserted into lumen 630. In the distal region which is bending, conventional stylet 620 contacts the lateral wall of the lumen in region 610.

FIG. 6B illustrates a lumen 605 of an electrode assembly while in its substantially straight configuration prior to implantation. An embodiment of a stylet 652 is inserted into lumen 630. In the distal region which is bending, each of the three regions 602, 604, 606 partially contact the lateral wall of the lumen to provide different degrees of resistance to the inherent bending force of the electrode assembly. Specifically, given the same materials and cross-sectional shape, region 602 has a larger cross-sectional area resulting in a substantial portion of its exterior surface contacting the surface of the lumen, while regions 604 and 606 have a smaller cross-sectional area and a smaller amount of surface area which contacts the lumen.

A suitable material for the stylet 40 is platinum, however, different materials could be chosen. Other suitable materials include titanium, platinum iridium, stainless steel or other metals, as well as polymers such as PTFE or polyurethane. When using metal, controlling the hardness of the material may be done by using cold working or annealing to temper the material.

In one form of construction, the features may be chemically etched from a larger stylet of diameter, for example, 0.15 mm.

Alternatively, the stylet could be a combination of the materials, for example a platinum core of about 0.1 mm diameter with material added to the larger portions e.g. platinum iridium. This example would increase the strength of those areas more than just keeping the same material.

In another aspect, the entire main body of the stylet could be provided with a substantially uniform cross section, that is thinner than prior art stylets, but which has a portion that is required to have greater bending strength, made from a more resilient or robust material. For example, region 3 could be made of the platinum iridium alloy, while the remainder of the main body could be pure platinum. Other variations of material could be provided to replace the thicker regions 2 and 4 as required.

It will also be appreciated that the stylet 40 could be considered part of the implant or be part of a surgical tool separate from the implant. For example, a stylet could be provided separate to a medical implant, for use by the surgeon during the implantation procedure. Alternatively, the medical implant, such as a cochlear implant, could be provided with the stylet for use by the surgeon during the implantation procedure of that cochlear implant.

It will also be appreciated that different geometric shapes to those shown in FIGS. 3A, 3B, 4 and 5 for the different regions could also be used. Including having regions within region 3, the widest region, that have a reduced diameter, to provide a “corrugated” or “notched” surface area to effectively further reduce the overall stylet withdrawal forces.

It will also be appreciated that alternative manufacturing methods could be used. For example, two separate stylets, joined together to make a change in geometric shape, and/or, change in material.

It will also be appreciated that the various aspects of the present invention can be used in combination with prior art arrangements which also address the issue of decreasing the withdrawal force and other movement restriction forces between the lumen and stylet, such as those described in US Patent Application No. 20070127745, and Teflon lumens as described in US Patent Application No. 2004/0220651, as well as other lumen arrangements such as those described in Australian Provisional Patent Application No. 2007906554 entitled “Cochlear Implant with Improved Lumen Arrangement”. Each of these documents has previously been incorporated by reference in its entirety.

The stylets produced according to the various aspects of the present invention may also be used with all suitable techniques of insertion, including the Standard Insertion Technique as described in U.S. Pat. No. 6,421,569, and the Advance Off Stylet insertion technique, as described in US 2004/0243212.

All documents, patents, journal articles and other materials cited in the present application are hereby incorporated by reference herein.

Although the present invention has been fully described in conjunction with several embodiments thereof with reference to the accompanying drawings, it is to be understood that various changes and modifications may be apparent to those skilled in the art. It should be understood that embodiments of the present invention may provide a combination of one or more of the above or other advantages, and that the disclosed embodiments need not provide each of the above advantages. It should also be understood that the present invention may be utilized in connection with any medical device now or later developed that may be implanted temporarily or permanently into a patient, or devices used in connection with the delivery or removal of devices, fluids, or other materials to or from a recipient. Such changes and modifications are to be understood as included within the scope of the present invention as defined by the appended claims, unless they depart therefrom.

What is claimed is:

1. An elongate stylet for removable insertion into a lumen of a stimulating medical implant, comprising:
a plurality of contiguous longitudinal regions, wherein a cross-sectional area of at least some of the regions are different.

2. The stylet of claim 1,
wherein the stimulating medical implant is a precurved elongate carrier member having contiguous longitudinal regions each of which have an inherent bending force,
wherein when the stylet is positioned in the lumen of the stimulating assembly, at least one region of the stylet is located in a corresponding one or more regions of the carrier member, and
wherein one or more of the at least one region of the stylet has a combination of cross-sectional area and material that provide a resistance to the bending force of the corresponding one or more regions of the carrier member.

3. The stylet of claim 2,
wherein the carrier member is configured to be implanted in a human cochlea, and
wherein the stylet is positioned in the lumen prior to implantation of the carrier member into the cochlea, and wherein the one or more regions of the carrier member corresponding to each of the at least one region of the stylet comprise one or more regions of the carrier member in which each of the corresponding at least one region of the stylet is positioned prior to implantation.

4. The stylet of claim 2,
wherein the bending force of one of the longitudinal regions of the carrier member is different that the bending force of another of the longitudinal regions of the carrier member.

5. The stylet of claim 2,
wherein the bending force of one of the longitudinal regions of the carrier member is approximately zero.

6. The stylet of claim 1, wherein the stylet further comprises transition regions between the neighboring regions of the plurality of longitudinal regions.

7. The stylet of claim 1,
wherein the stimulating medical implant is an elongate stimulating assembly having contiguous longitudinal regions,
wherein when the stylet is positioned in the lumen of the stimulating assembly, at least one region of the stylet is located in a corresponding one or more regions of the stimulating assembly, and
wherein one or more of the at least one region of the stylet has a cross-sectional area that provides a desired amount of at least one of static and frictional force between the stylet and lumen.

8. The stylet of claim 7,
wherein a plurality of the at least one region of the stylet have cross-sectional areas that provides a desired total amount of at least one of static and dynamic friction force between the stylet and lumen.

9. The stylet of claim 1, wherein the stimulating medical implant is a precurved elongate stimulating assembly configured to be implanted into a cochlea, and further wherein the cross-sectional area and length of each of said regions are such that the stylet substantially resists bending forces of a first region that the main body of the stylet is maximised at a region of the stylet that requires the greatest bending strength relative to other regions of the stylet.

10. The stylet of claim 1, wherein the stimulating medical implant is a precurved elongate stimulating assembly configured to be implanted into a cochlea, and further wherein the cross-sectional area and length of said regions are such that the stylet substantially resists bending forces of a first region that the main body of the stylet is maximised at a region of the stylet that requires the greatest bending strength relative to other regions of the stylet.

11. The stylet of claim 1, wherein a first of said plurality of regions between about 0 mm and about 2 mm from the tip has a diameter of about 0.1 mm.

12. The stylet of claim 11, wherein a second region of the stylet between about 4 mm and about 6 mm from the tip has a diameter of about 0.125 mm.

13. The stylet of claim 12, wherein a third region of the stylet between about 8 mm and about 12 mm from the tip has a diameter of about 0.15 mm.

14. The stylet of claim 13, wherein a fourth region of the stylet between about 15 mm and about 34.5 mm from the tip has a diameter of about 0.125 mm.

15. The stylet of claim 14, wherein there is provided a first transition region between the first region and the second region; a second transition region between the second region and the third region; and a third transition region between the third region and the fourth region.

16. A stylet comprising a handle, a tip, and a main body therebetween, for use in a medical implant, wherein a cross sectional area of the main body of the stylet is reduced at a region of the stylet that requires less bending strength than other regions of the stylet.

17. A kit comprising:
a stimulating medical implant having a lumen; and
an elongate stylet for removable insertion into a lumen of the stimulating medical implant, the stylet having a plurality of contiguous longitudinal regions, wherein a cross-sectional area of at least some of the regions are different.

18. The medical implant of claim 17, wherein the medical implant is a cochlear implant.

19. The kit of claim 17,
wherein the stimulating medical implant is a precurved elongate carrier member having contiguous longitudinal regions each of which have an inherent bending force,
wherein when the stylet is positioned in the lumen of the stimulating assembly, at least one region of the stylet is located in a corresponding one or more regions of the carrier member, and
wherein one or more of the at least one region of the stylet has a combination of cross-sectional area and material that provide a resistance to the bending force of the corresponding one or more regions of the carrier member.

20. The kit of claim 19,
wherein the carrier member is configured to be implanted in a human cochlea, and
wherein the stylet is positioned in the lumen prior to implantation of the carrier member into the cochlea, and wherein the one or more regions of the carrier member corresponding to each of the at least one region of the stylet comprise one or more regions of the carrier member in which each of the corresponding at least one region of the stylet is positioned prior to implantation.

21. The stylet of claim 17,
wherein the bending force of one of the longitudinal regions of the carrier member is different that the bending force of another of the longitudinal regions of the carrier member.

22. The stylet of claim 17,
wherein the stimulating medical implant is an elongate stimulating assembly having contiguous longitudinal regions,
wherein when the stylet is positioned in the lumen of the stimulating assembly, at least one region of the stylet is located in a corresponding one or more regions of the stimulating assembly, and
wherein one or more of the at least one region of the stylet has a cross-sectional area that provides a desired amount of at least one of static and frictional force between the stylet and lumen.
